(19) United States
(12) Patent Application Publication
(10) Pub. No.: US 2009/0111168 A1
(43) Pub. Date: Apr. 30, 2009

(54) BIOCHIP AND METHOD OF FABRICATION

(75) Inventors: Won-Sun KIM, Gyeonggi-do (KR); Jung-Hwan HAH, Gyeonggi-do (KR); Sung-Min CHI, Gyeonggi-do (KR); Sun-Ok JUNG, Gyeonggi-do (KR); Man-Hyoung RYOO, Gyeonggi-do (KR)

Correspondence Address: MARGER JOHNSON & MCCOLLOM, P.C., 210 SW MORRISON STREET, SUITE 400, PORTLAND, OR, 97204, US

(73) Assignee: SAMSUNG ELECTRONICS CO., LTD., Gyeonggi-do (KR)

(21) Appl. No.: 12/198,672

(22) Filed: Aug. 26, 2008

(30) Foreign Application Priority Data
Aug. 27, 2007 (KR) 10-2007-0086289

(51) Int. Cl. C12M 1/34

(52) U.S. Cl. 435/287.1

(57) ABSTRACT

A biochip and method of fabricating the same are provided. The biochip can include a substrate, a capping layer pattern partially covering a top surface of the substrate, and a plurality of probes coupled to the top surface of the substrate exposed by the capping layer pattern.

### CROSS-REFERENCE TO RELATED APPLICATION

This application claims priority to Korean Patent Application No. 10-2007-0086289, filed Aug. 27, 2007, the disclosure of which is incorporated herein by reference in its entirety.

### BACKGROUND

The disclosed technology relates to a biochip and method of fabricating the same, and more particularly, to a biochip for analyzing components of a bio sample using probes, and method of fabricating the same.

### SUMMARY

The disclosed technology provides a biochip having improved analysis reliability.

The disclosed technology also provides a method of fabricating a biochip having improved analysis reliability.

The above and other objects of the disclosed technology will be described in or be apparent from the following description of various embodiments.

Certain embodiments provide a biochip including a substrate, a capping layer pattern partially covering a top surface of the substrate, and a plurality of probes coupled to the top surface of the substrate exposed by the capping layer pattern.

Other embodiments provide a biochip comprising a substrate including a first region and a second region, an active layer formed on the substrate, and a probe cell isolating pattern formed on the active layer, wherein the probe cell isolating pattern is positioned on the second region.

Still other embodiments provide a biochip including a substrate including a first region and a second region, a probe cell isolating pattern formed on the second region of the substrate, and an active pattern formed on the first region of the substrate on the active layer, wherein a thickness of the active pattern is smaller than that of the probe cell isolating pattern.

Further embodiments provide a method of fabricating a biochip including forming an active layer on an entire top surface of a substrate, forming a capping layer pattern partially covering the active layer on the active layer, and coupling a plurality of probes to the active layer exposed by the capping layer pattern.

Yet other embodiments provide a method of fabricating a biochip including forming a capping layer pattern partially covering a substrate on a top surface of the substrate, forming an active pattern on the top surface of the substrate exposed by the capping layer pattern, and coupling a plurality of probes to the active pattern.

### BRIEF DESCRIPTION OF THE DRAWINGS

The above and other features and advantages of the disclosed technology will become more apparent by describing in detail various embodiments thereof with reference to the attached drawings in which:

FIG. 1 is a layout view of biochips according to certain embodiments of the disclosed technology;

FIGS. 2 through 7 are sectional views of biochips according to various embodiments of the disclosed technology;

FIGS. 8A through 8E are sectional views illustrating a method of fabricating biochips according to certain embodiments of the disclosed technology; and

FIGS. 9A through 9D are sectional views illustrating a method of fabricating biochips according to other embodiments of the disclosed technology.

### DETAILED DESCRIPTION

Advantages and features of the disclosed technology and methods of accomplishing the same may be understood more readily by reference to the following detailed description of various embodiments and the accompanying drawings. The disclosed technology may, however, be embodied in many different forms and should not be construed as being limited to the embodiments set forth herein. Rather, these embodiments are provided so that this disclosure will be thorough and complete and will fully convey various concepts of the disclosed technology to those skilled in the art, and the present invention will only be defined by the appended claims.

Accordingly, in some specific embodiments, well known materials or methods have not been described in detail in order to avoid obscuring the invention.

It is noted that the use of any and all examples, or exemplary terms provided herein is intended merely to better illuminate the invention and is not a limitation on the scope of the invention unless otherwise specified. As used herein, the singular forms are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The disclosed technology will be described with reference to perspective views, cross-sectional views, and/or plan views, in which various embodiments of the disclosed technology are shown. Thus, the profile of an exemplary view may be modified according to manufacturing techniques and/or allowances. That is, the embodiments of the disclosed technology are not intended to limit the scope of the present invention but cover all changes and modifications that can be caused due to a change in manufacturing process. In the drawings, various components may be exaggerated or reduced for clarity. Like reference numerals refer to like elements throughout the specification.

Biochips according to certain embodiments of the disclosed technology analyze biomolecules contained in biological samples and are used in gene expression profiling, genotyping through detection of mutation or polymorphism such as Single-Nucleotide Polymorphism (SNP), a protein or peptide assay, potential drug screening, development and preparation of novel drugs, etc. Biochips employ appropriate probes according to the kind of biological sample to be analyzed. Examples of probes useful for biosensors include a DNA probe, a protein probe such as an antibody/antigen or a bacteriorhodopsin, a bacterial probe, a neuron probe, etc. A biosensor fabricated in the form of a chip may also be referred to as a biochip. For example, according to the kind of probe used, the biosensor may be referred to as a DNA chip, a protein chip, a cellular chip, a neuron chip, etc.

Biochips according to certain embodiments of the disclosed technology may comprise oligomer probes, suggesting that the number of monomers contained in the oligomer probe is in an oligomer level. Here, the oligomer has a molecular weight of about 1,000 or less but the disclosed technology is not limited thereto. The oligomer may include about 2-500 monomers, preferably 5-30 monomers. However, the characteristics of the oligomer probe are not limited to the ranges listed above.

The monomers constructing an oligomer probe may vary according to the type of biological sample to be analyzed, and examples thereof include nucleosides, nucleotides, amino acids, peptides, etc.

As used herein, the terms “nucleosides” and “nucleotides” include not only known purine and pyrimidine bases, but also methylated purines or pyrimidines, acylated purines or pyrimidines, etc. Furthermore, the “nucleosides” and “nucleotides” include not only known (deoxy)ribose, but also a modified sugar which contains a substitution of a halogen atom or an aliphatic group for at least one hydroxyl group or is functionalized with ether, amine, or the like.

As used herein, the term “amino acids” are intended to refer to not only naturally occurring, L-, D-, and nonchiral amino acids, but also modified amino acids, amino acid analogs, etc.

As used herein, the term “peptides” refer to compounds produced by an amide bond between the carboxyl group of one amino acid and the amino group of another amino acid.

Unless otherwise specified in the following exemplary embodiments, the term “probe” is a DNA probe, which is an oligomer probe consisting of about 5-30 covalently bound monomers.

However, the disclosed technology is not limited to the probes listed above and a variety of probes may used.

Embodiment of the disclosed technology will now be described with reference to the accompanying drawings.

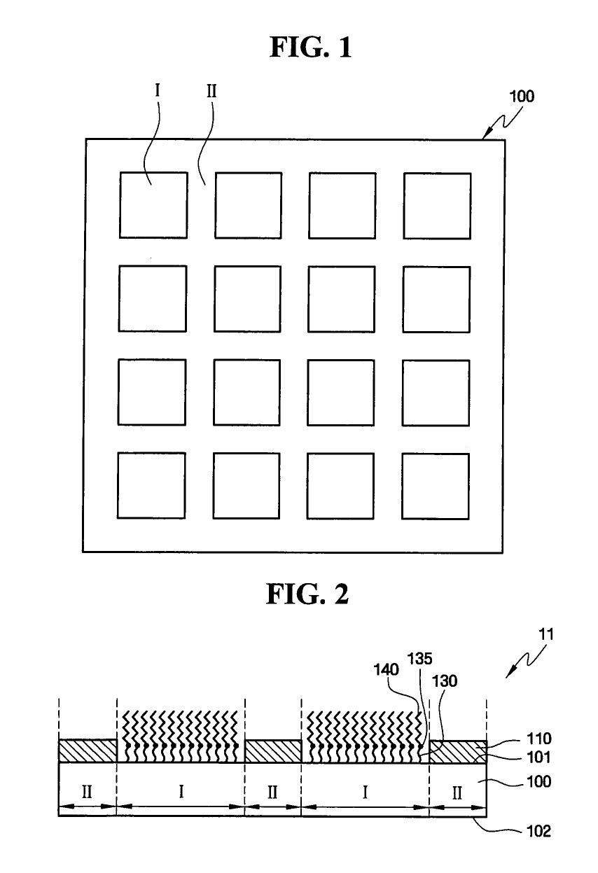

FIG. 1 is a layout view of biochips according to certain embodiments of the disclosed technology.

Referring to FIG. 1, a substrate 100 for a biochip according to an exemplary embodiment of the disclosed technology includes a plurality of probe cell regions I and non-probe cell regions II. The probe cell regions I and the non-probe cell regions II are defined according to the presence or absence of probes 140 to be coupled. In other words, the probe cell region I of the substrate 100 is a region of the substrate 100 on which a plurality of probes 140 are coupled, while the non-probe cell region II 100 is a region of the substrate 100 on which the probes 140 are not coupled. Probe cells including the coupled plurality of probes 140 are formed on the probe cell regions I of the substrate 100.

While the probes 140 of the same sequences are immobilized on a probe cell region I, the probes 140 of different sequences may be immobilized on different probe cell regions I.

The different probe cell regions I are separated from each other by the non-probe cell region II. Thus, each probe cell regions I is surrounded by the non-probe cell regions II. The plurality of probe cell regions I may be arranged in a matrix configuration. Here, the matrix configuration does not necessarily have a regular pitch.

Unlike the independent probe cell regions I, the non-probe cell regions II may be connected to one another into a single unit. For example, the non-probe cell regions II are arranged in a lattice configuration.

FIG. 2 is a sectional view of a biochip 11 according to an embodiment of the disclosed technology.

Referring to FIG. 2, the biochip 11 according to an embodiment of the disclosed technology includes a substrate 100, a capping layer pattern 110, and a plurality of probes 140.

The substrate 100 is a base for coupling of the plurality of probes 140, and may be made of various materials. For example, the substrate 100 may be either a flexible material or a rigid substrate. In addition, the substrate 100 may be either an opaque substrate or a transparent substrate. The flexible substrate may be a membrane of nylon or cellulose, or a plastic film. The rigid substrate may be a semiconductor substrate made of silicon, or a transparent substrate made of quartz, or soda-lime glass. Among the illustrated examples, when a transparent substrate is used, it can be advantageously compatible with substrates that have been widely used for various known applications, including relatively thin slide substrates for use in, for example, microscopic observation, relatively thick, large-screen liquid crystal display (LCD) panels, etc.

The capping layer pattern 110 is formed on a top surface 101 of the substrate 100, partially covering the top surface 101. Here, the phrase “partially covering the top surface 101” is intended to encompass a meaning of partially exposing the top surface 101. For example, when the capping layer pattern 110 is formed on the non-probe cell regions II of the substrate 100, the probe cell regions I of the substrate 100 are exposed. In certain embodiments of the disclosed technology, the capping layer pattern 110 completely overlaps with the non-probe cell regions II. In other words, the capping layer pattern 110 may have the same shape as the non-probe cell regions II. In this case, it is understood that the probe cell regions I and the non-probe cell regions II are divided by the capping layer pattern 110. In other words, the substrate 100 is divided into the probe cell regions I and the non-probe cell regions II according to whether the capping layer pattern 110 is formed on the substrate 100 or the substrate 100 is exposed.

The capping layer pattern 110 is formed on the non-probe cell regions II and is preferably not coupled with the probes 140. In order to prevent the capping layer pattern 110 from being coupled with the probes 140, the capping layer pattern 110 may be made of materials without functional groups capable of being coupled with the linkers 130 and the plurality of probes 140. Examples of the functional groups include a metallic film, a metallic nitride film, a silicon nitride film, and the like. Examples of the useful metal include Ti, Ta, Cr, Al, Cu, Au, Ag, or alloys of these metals. In non-limiting exemplary embodiments, a Ti film or a TaN film is used to form the capping layer pattern 110.

In certain embodiments of the disclosed technology, the capping layer pattern 110 may be formed of a single film made of at least one of the aforementioned materials.

As described above, the capping layer pattern 110 prevents undesired coupling of the linkers 130 and the plurality of probes 140 on the non-probe cell regions II. Thus, there is no limitation in the thickness of the capping layer pattern 110. In other words, as long as the capping layer pattern 110 is thick enough to cover the functional groups on the top surface 101 of the substrate 100, the coupling of the linkers 130 and the plurality of probes 140 can be effectively prevented. To ensure reliable shapes of patterns, the capping layer pattern 110 may have an average thickness of about 200 Å. In addition, in order to allow a biological sample to be sufficiently wet into probe cells during hybridization, that is, in order to prevent spreadability of the biological sample from being interfered by the capping layer pattern 110, the capping layer pattern 110 may have a thickness of about 1000 Å or less.

The linkers 130 are formed on the top surface 101 of the substrate 100 in the probe cell regions I exposed by the capping layer pattern 110. The linkers 130 contain functional groups 135 each having a first end coupled to the top surface 101 of the substrate 100 and a second end coupled to the probes 140. When the probes 140 are DNA probes (i.e., oligo nucleotide probes), examples of the functional groups 135 that can be coupled to the probes 140 include hydroxyl groups, aldehyde groups, carboxyl groups, amino groups, amide groups, thiol groups, halo groups, and sulfonate groups.

When the substrate 100 is made of a material containing a Si(OH) group, e.g., glass, the linkers 130 may include a silicon group capable of producing siloxane (Si—O) bonds with the Si(OH) group. Examples of materials used for the linkers 130, including the silicon group as well as the functional groups 135 that can be coupled to the probes 140 (hereinafter probes include monomers for probes), include N-(3-(triethoxysilyl)-propyl)-4-hydroxybutyramide, N,N-bis(hydroxyethyl)aminopropyl-triethoxysilane, acetoxypropyl-triethoxysilane, 3-Glycidoxy propyltrimethoxysilane), and silicon compounds disclosed in PCT application WO 00/21967, the content of which are hereby incorporated by reference.

The plurality of probes 140 are immobilized on the top surface 101 through the linkers 130. That is, in the probe cell regions I, the plurality of probes 140 are coupled to the second ends of the linkers 130.

As described above, the biochip 11 according to the current embodiment includes a capping layer pattern 110 formed on the non-probe cell regions II of the substrate 100, and the linkers 130 and the plurality of probes 140 formed on the probe cell regions I of the substrate 100.

When the top surface 101 of the substrate 100 includes the functional groups 135 capable of coupling with the plurality of probes 140, the linkers 130 may be omitted.

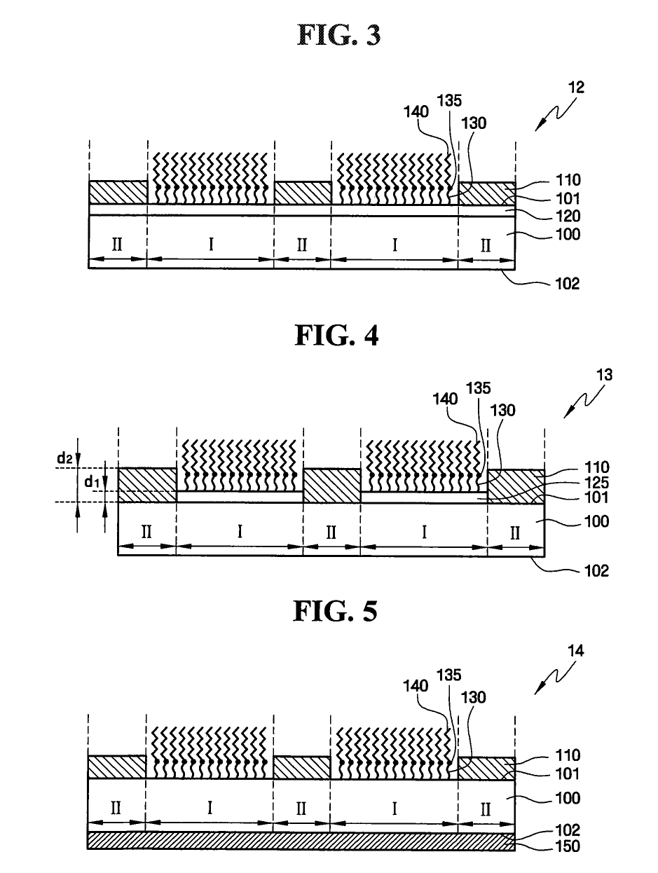

FIG. 3 is a sectional view of a biochip 12 according to still another embodiment of the disclosed technology.

Referring to FIG. 3, a biochip 12 according to the current embodiment of the disclosed technology further includes an active layer 120 formed on the top surface 101. The active layer 120 may be formed on substantially the entire top surface 101 of the substrate 100, regardless of whether it is in either the probe cell regions I or the non-probe cell regions II. The linkers 130 are formed on the active layer 120. The surface of the active layer 120 contains functional groups capable of being coupled to the linkers 130 and/or the probes 140. When the top surface 101 is not coupled to the linkers 130 and/or the probes 140, or when there are negligible functional groups coupled to the linkers 130 and/or the probes 140, the active layer 120 may be advantageously provided.

The capping layer pattern 110 is formed on the active layer 120. A material of the capping layer pattern 110 is substantially the same as above with reference to FIG. 2, and a repeated explanation will not be given. Thus, the linkers 130 are selectively formed on the active layer 120 exposed by the capping layer pattern 110, that is, on the active layer 120 in the probe cell regions I.

Further, the active layer 120 may be formed of a material that is substantially stable against hydrolysis upon a hybridization assay, e.g., upon contact with a pH 6-9 phosphate or TRIS buffer. From this standpoint, the active layer 120 is preferably made of a silicon oxide film such as a PE-TEOS film, a HDP oxide film, a P—SiH₄ oxide film or a thermal oxide film; silicate such as hafnium silicate or zirconium silicate; a metallic oxynitride film such as a silicon nitride film, a silicon oxynitride film, a hafnium oxynitride film or a zirconium oxynitride film; a metal oxide film such as ITO; a metal such as gold, silver, copper or palladium; polyimide; polyamine; or polymers such as polystyrene, polyacrylate or polyvinyl.

A surface of the active layer 120 may have a predetermined degree of roughness in order to ensure sufficient space for coupling with the linkers 130. For example, when the active layer 120 is formed of a thermal oxide film, it may have surface roughness of about 5 nm to about 100 nm.

The linkers 130 may be formed of a material containing functional groups 135 each having a first end coupled to a top surface of the active layer 120 and a second end coupled to the probes 140. The material forming the linkers 130 may vary according to the material forming the active layer 120. When the active layer 120 is made of, for example, a silicon oxide film, silicate or a silicon oxynitride film, the linkers 130 may contain a silicon group capable of reacting with Si(OH) groups on the surface of the active layer 120 to produce siloxane (Si—O) bonds. Examples of useful materials are the same as described above with reference to FIG. 2.

When the active layer 120 is made of a metal oxide film, the functional group at one end of each of the linkers 130 coupled to the active layer 120 may include a metal alkoxide or metal carboxylate group. When the active layer 120 is made of a silicon nitride film, a silicon oxynitride film, a metallic oxynitride film, polyimide, or polyamine, the functional group at one end of each of the linkers 130 coupled to the active layer 120 may include anhydrides, acid chlorides, alkyl halides, or chlorocarbonates. When the active layer 120 is made of a polymer, the functional group at one end of each of the linkers 130 coupled to the active layer 120 may include an acrylic, styryl, or vinyl group.

The plurality of probes 140 are coupled to the active layer 120 of the top surface 101 via the linkers 130. That is, the plurality of probes 140 are coupled to the second ends of the linkers 130 in the probe cell regions I to form probe cells. As described above, since the linkers 130 are formed only on the probe cell regions I, the probes 140 are selectively coupled to only the probe cell regions I.

As described above, the biochip 12 according to the current embodiment includes the active layer 120 and the capping layer pattern 110 formed on the non-probe cell regions II of the substrate 100, and the active layer 120, the linkers 130 and the plurality of probes 140 formed on the probe cell regions I of the substrate 100.

Meanwhile, when the surface of the active layer 120 contains functional groups capable of being coupled to the probes 140, the linkers 130 may be omitted.

FIG. 4 is a sectional view of a biochip 13 according to yet another embodiment of the disclosed technology.

Referring to FIG. 4, the biochip 13 according to the current embodiment of the disclosed technology further includes active patterns 125 formed on the top surface 101. The active patterns 125 are different from the active layer 120 shown in FIG. 3 in that they are selectively formed only on the probe cell regions I. The capping layer pattern 110 may contribute to selectively forming the active patterns 125 only on the top surface 101 of the substrate 100. When the active patterns 125 are formed of, for example, a thermal oxide film, the forming of the capping layer pattern 110 is followed by performing annealing, thereby selectively forming the thermal oxide film only on the probe cell regions I of the substrate 100 exposed by the capping layer pattern 110.

Each of the active patterns 125 correspond to the probe cell region I, and the active patterns 125 are physically discrete from each other. Materials forming the active patterns 125 are substantially the same as those of the active layer 120 shown in FIG. 3. According to some exemplary embodiments of the disclosed technology, a thickness d1 of each of the active patterns 125 is smaller than a thickness d2 of the capping layer pattern 110. Accordingly, the top surface of the active patterns 125 is lower than that of the capping layer pattern 110. Although not shown in the drawings, according to other embodiments of the disclosed technology, the thickness of each of the active patterns 125 may be greater than that of the capping layer pattern 110. In such cases, the top surface of the active patterns 125 may be higher than that of the capping layer pattern 110. Meanwhile, in the embodiment shown in FIG. 3, in which the capping layer pattern 110 is formed on the active layer 120, the top surface of the active layer 120 is necessarily higher than that of the capping layer pattern 110.

As described above, the biochip 13 according to the current embodiment includes a capping layer pattern 110 formed on the non-probe cell regions II of the substrate 100, and the active patterns 125, the linkers 130 and the plurality of probes 140 formed on the probe cell regions I of the substrate 100.

When the surface of the active patterns 125 includes functional groups capable of coupling with the plurality of probes 140, the linkers 130 may be omitted.

FIGS. 5 through 7 are sectional views of biochips 14 through 16 according to still other embodiments of the disclosed technology, illustrating modifications of the biochips 11 through 13 shown in FIGS. 2 through 4, each further including a bottom capping layer 150.

The bottom capping layer 150 is formed on substantially the entire bottom surface 102 opposite to the top surface 101 of the substrate 100. In a case where the bottom surface 102 contains functional groups capable of being coupled to the linkers 130 or the probes 140, the bottom capping layer 150 prevents undesired coupling which may be caused by the functional groups by preventing exposure of the functional groups. Accordingly, data noise can be avoided, thereby increasing the analysis reliability.

From this standpoint, the bottom capping layer 150 is made of a material without functional groups capable of being coupled to the linkers 130 or the probes 140. Thus, the material useful for forming the capping layer pattern 110 can be used. However, the material forming the capping layer pattern 110 and that forming the bottom capping layer 150 are not necessarily the same materials. In non-limiting exemplary embodiments, a Ti film or a TaN film is used as the bottom capping layer 150.

In certain embodiments of the disclosed technology, when a transparent substrate is used as the substrate 100, the bottom capping layer 150 has reflectivity along with the aforementioned functions and increases analysis efficiency during fluorescence detection. To ensure sufficiently high data analysis efficiency, the reflectivity of the bottom capping layer 150 is preferably about 20% or higher.

A thickness of the bottom capping layer 150 is related with the reliability of a capping ability and the efficacy of a reflecting ability. For satisfactory capping and reflecting abilities, the bottom capping layer 150 preferably has a thickness in a range of about 1000 to about 3000 Å.

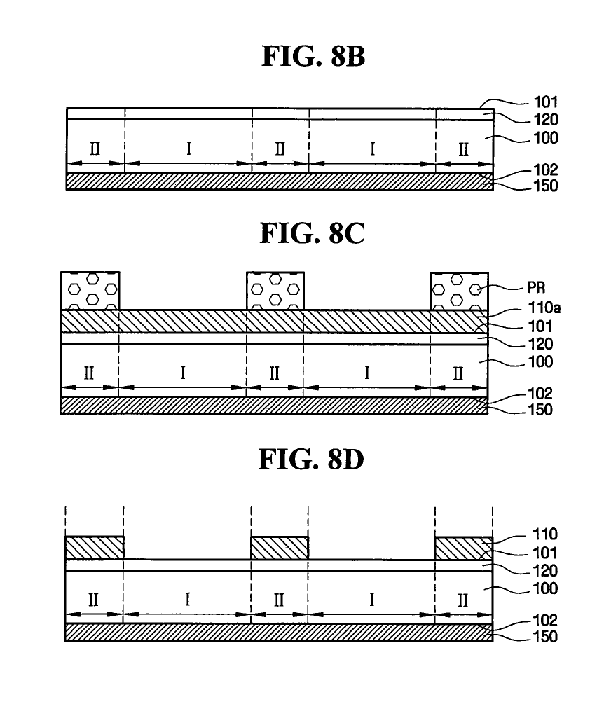

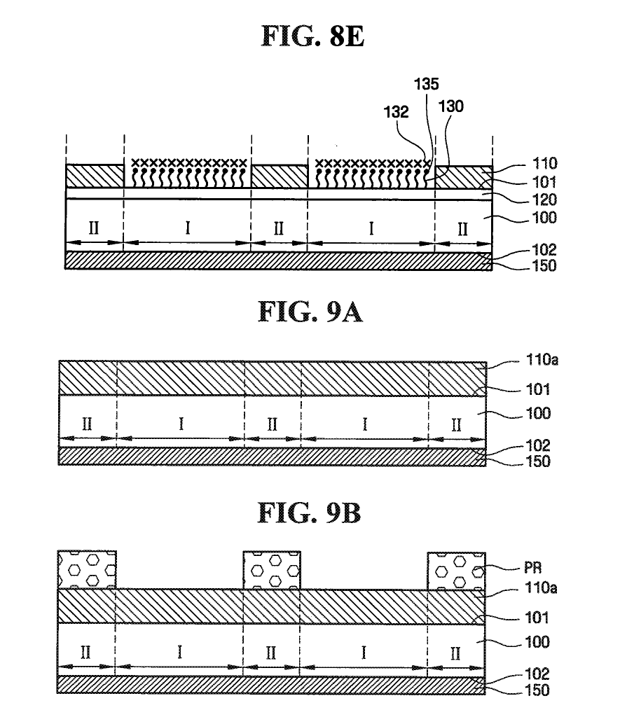

Hereinafter, methods of fabricating biochips according to some exemplary embodiments of the disclosed technology will be described. FIGS. 8A through 8E are sectional views illustrating a method of fabricating biochips according to certain embodiments of the disclosed technology.

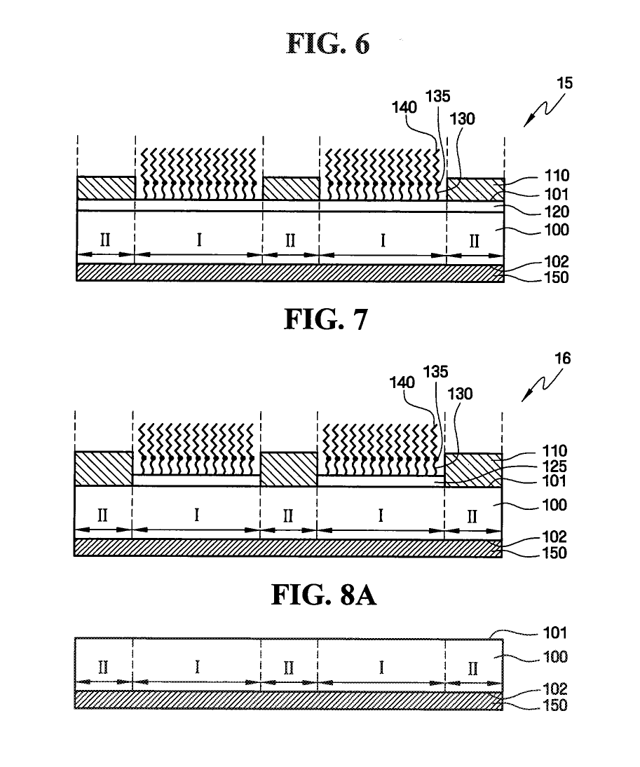

The methods of fabricating biochips according to some exemplary embodiments of the disclosed technology will be described by way of the biochip 15 shown in FIG. 6, with reference to FIGS. 8A through 8E.

Referring to FIG. 8A, a substrate 100 including a probe cell region I and a non-probe cell region II is provided. Next, a bottom capping layer 150 is formed on a bottom surface 102 of the substrate 100. The bottom capping layer 150 may be formed by a widely known deposition process including, for example, plasma enhanced chemical vapor deposition (PECVD), metal organic chemical vapor deposition (MOCVD), atomic layer deposition (ALD), plasma enhanced atomic layer deposition (PEALD), and the like.

Referring to FIG. 8B, an active layer 120 is formed on the top surface 101 of the substrate 100. The active layer 120 is formed by, for example, various deposition processes well-known in the art or a thermal oxidation process. When the thermal oxidation process is employed, the substrate 100 is annealed at a temperature in a range of about 900 to about 1200° C. for about 3 to about 12 hours. Here, since the bottom surface 102 is protected by the bottom capping layer 150, the active layer 120 formed of a thermal oxide film is selectively formed only on the top surface 101 of the substrate 100. The formed active layer 120 may have a surface roughness of about 5 nm to about 100 nm.

Referring to FIG. 8C, an upper capping layer 110a is formed on the active layer 120. The upper capping layer 110a is formed in the same manner as the bottom capping layer 150. To avoid loss of the bottom capping layer 150 during etching of the upper capping layer 110a, which will later be described, the upper capping layer 110a is preferably made of a material having different etching selectivity from that of the bottom capping layer 150. However, in a case where there is no risk of the bottom capping layer 150 being exposed to an etchant, such as in a case of employing an apparatus or structure of preventing the etchant from penetrating into the bottom surface 102 of the substrate 100 during the etching of the upper capping layer 110a, the upper capping layer 110a may be made of the same material as the bottom capping layer 150. Subsequently, photoresist patterns PR opening probe cell regions I are formed on the upper capping layer 110a.

Referring to FIG. 8D, the upper capping layer 110a is etched using the photoresist patterns PR as etching masks to form the capping layer pattern 110. The etching may be performed by anisotropic etching using a drying etchant. However, a wet etching process using a dry etchant or a wet etchant may also be employed. Upon etching, the upper capping layer 110a in the probe cell regions I is removed to expose the active layer 120. However, the active layer 120 in the non-probe cell regions II of the substrate 100 is still covered and protected by the capping layer pattern 110. Next, the photoresist patterns PR are removed.

Referring to FIG. 8E, optionally, in order to modify the surface of the active layer 120 so as to facilitate a reaction between the active layer 120 and the linkers 130, the active layer 120 is subjected to a surface treatment such as ozonolysis, acid treatment, or base treatment, using, for example, a Piranha solution (a mixture of sulfuric acid and hydrogen peroxide), a hydrofluoric acid solution, an ammonium hydroxide solution, or O₂ plasma.

Subsequently, the linkers 130 are formed on the surface treated active layer 120. Here, in a case where the probes 140 are synthesized using a photolithography process, which will be described below, photolabile groups 132 are attached to functional groups 135 of the linkers 130. The linkers 130 are selectively formed only on the active layer 120 in the probe cell regions I, while they are not formed on the active layer 120 in the non-probe cell regions II, which is protected by the capping layer pattern 110.

Next, the probes 140 are formed on the linkers 130. When the functional groups 135 capable of coupling with the probes at second ends of the linkers 130 are protected by the photolabile groups 132, selective exposure by probe cell regions I is performed to remove the photolabile groups 132 and the probes 140 are then coupled to the second ends of the linkers 130. For example, the coupling of the probes 140 may be performed by spotting onto completed probes, or synthesizing monomers for probes (e.g., nucleotide phosphoamidite monomers having functional groups protected by photolabile groups) by photolithography. After forming the probes 140, the biochip 15 shown in FIG. 6 is completed.

In certain embodiments of the disclosed technology (e.g., in the embodiment shown in FIG. 3), in which it is not necessary or intended to increase reflectivity of a biochip, as in a case where an analysis scheme other than fluorescence analysis may be employed, where a scanning method other than the use of the scanner illustrated above may be employed, or where an opaque substrate may be used, removal of the bottom capping layer 150 may further be performed. The removing of the bottom capping layer 150 is carried out using, for example, a Piranha solution, or other cleaning solutions or a wet etching solution.

In more detail, if the bottom surface 102 remains unreacted with the linkers 130, if the reactivity with the linkers 130 is insignificant, or if in the forming of the linkers 130 the linkers 130 are drastically prohibited from being provided to the bottom surface 102, then the bottom capping layer 150, which has already contributed in preventing the active layer 120 from being formed on the bottom surface 102, may be removed after steps shown in FIG. 8. If the bottom capping layer 150 is removed, the bottom surface 102 is exposed. Thus, even after the subsequent steps including coating, synthesis, exposure, scanning, etc, it is not necessary to replace equipment due to a defect occurring in the equipment or a dimension error. In an alternative embodiment of the disclosed technology, removal of the bottom capping layer 150 may be performed after the coupling of the probes 140.

To fabricate the biochip 11 shown in FIG. 2 or the biochip 14 shown in FIG. 5, some of the above-described steps may be modified. For example, to fabricate the biochip 14 shown in FIG. 5, the steps shown in FIG. 8 are skipped. Further, to fabricate the biochip 11 shown in FIG. 2, the steps for forming the bottom capping layer 150 may be skipped or the steps for removing the bottom capping layer 150 may be further performed.

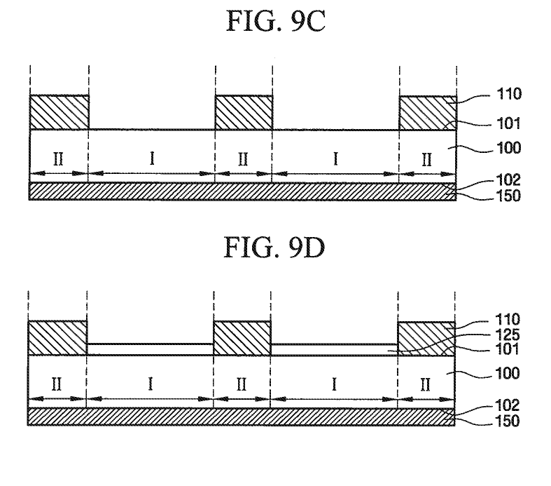

FIGS. 9A through 9D are sectional views illustrating a method of fabricating biochips according to other embodiments of the disclosed technology, illustrating intermediate structures of the biochip shown in FIG. 7 by way of example.

In the method of fabricating the biochip according to the current embodiment of the disclosed technology, the same steps as shown in FIG. 8A are performed until the bottom capping layer 150 is formed on the bottom surface 102. Next, referring to FIG. 9A, the upper capping layer 110a is formed on substantially the entire top surface 101.

Referring to FIG. 9B, photoresist patterns PR opening the probe cell regions I are formed on the upper capping layer 110a.

Referring to FIG. 9C, the upper capping layer 110a is etched using the photoresist patterns PR as etching masks to form the capping layer pattern 110, which is performed in the same manner as in FIG. 8D. Since the capping layer pattern 110 is formed, the upper capping layer 110a in the probe cell regions I of the substrate 100 is removed to expose the top surface 101 of the substrate 100. However, the non-probe cell regions II of the substrate 100 are still covered and protected by the capping layer pattern 110.

Referring to FIG. 9D, the active patterns 125 are formed on the probe cell regions I of the exposed substrate. The active patterns 125 are formed by, for example, a thermal oxidation process. Here, since the top surface 101 is protected by the capping layer pattern 110 in the non-probe cell regions II, no thermal oxide film is formed thereon, and a thermal oxide film is selectively formed only on the top surface 101 in the probe cell regions I. Also, since the bottom surface 102 is protected by the bottom capping layer 150, it is also possible to prevent an unwanted thermal oxide film from being formed on the bottom surface 102.

Subsequent steps are substantially the same as those illustrated in FIG. 8E and subsequent figures.

Meanwhile, to fabricate the biochip 13 shown in FIG. 4, the steps for forming the bottom capping layer 150 may be skipped or the steps for removing the bottom capping layer 150 may be further performed. More details can be deduced from the description having been made above, and a detailed explanation will not be given.

The disclosed technology will be described in detail through the following concrete experimental examples. However, the experimental example is for illustrative purposes and other examples and applications can be readily envisioned by a person of ordinary skill in the art. Since a person skilled in the art can sufficiently analogize the technical contents which are not described in the following concrete experimental examples, the description thereabout is omitted.

### EXPERIMENTAL EXAMPLE 1

A Ti film was deposited to a thickness of 2000 Å on a bottom surface of a glass substrate using a CVD process. Then, the Ti film was baked at 1000° C. for 5 hours to form an active layer formed of a thermal oxide film having a thickness of about 5000 Å and a surface roughness of about 10 nm on a top surface of the glass substrate.

Subsequently, a TaN film was deposited on the thermal oxide film to a thickness of about 500 Å using a chemical vapor deposition (CVD) process. Next, a photoresist film was formed on the TaN film to a thickness of about 3.0 μm using a spin-coating process and baked at 100° C. for 60 seconds. The photoresist film was exposed to light using a checkerboard type dark tone mask with a pitch of 1.0 μm in a 365 nm-wavelength projection exposure machine and developed with a 2.38% TetraMethylAmmonium Hydroxide (TMAH) solution to form checkerboard type photoresist patterns to expose rectangular regions (probe cell regions) defined in the form of intersecting stripes. Subsequently, the TaN film was etched using the photoresist patterns as etching masks, thereby exposing the surface of the active layer corresponding to the respective probe cell regions.

Next, the Ti film was removed from the bottom surface of the glass substrate using a piranha solution (7:3 concentrated H₂SO₄/H₂O₂) and functional groups on the active pattern surface were activated.

Next, the active layer was spin-coated with bis(hydroxyethyl)aminopropyltriethoxysilane at 500 rpm for 30 seconds, and stabilized at room temperature for about 5 to 30 minutes. Then, the resultant product was treated with an acetonitrile solution containing NNPOC-tetraethyleneglycol and tetrazole (1:1) so that phosphoramidite protected with photolabile groups was coupled to the active patterns, and then acetyl-capped, which resulted in completion of protected linker structures.

Next, the probe cell regions were exposed to light using a binary chrome mask exposing desired active layer in a 365 nm-wavelength projection exposure machine with an energy of 1000 mJ/cm² for one minute to deprotect terminating functional groups of the linker structures. Then, the probe cell regions were treated with an acetonitrile solution containing amidite-activated nucleotide and tetrazole (1:1) to achieve coupling of the protected nucleotide monomers to the deprotected linker structures, and then treated with a THF solution (acetic anhydride (Ac20)/pyridine (py)/methylimidazole=1:1:1) and a 0.02M iodine-THF solution to perform capping and oxidation.

The above-described deprotection, coupling, capping, and oxidation processes were repeated to synthesize oligonucleotide probes having different sequences for the active layer in each probe cell region.

### EXPERIMENTAL EXAMPLE 2

A biochip was completed in the same manner as in Experimental Example 1, except that prior to formation of the active layer, a TaN film was formed in the same manner as in Experimental Example 1 and active patterns formed of a thermal oxidation film are formed on a top surface of a substrate exposed by the TaN film.

In some embodiments, a method of fabricating a biochip can include forming an active layer on an entire top surface of a substrate, forming a capping layer pattern partially covering the active layer on the active layer, and coupling a plurality of probes to the active layer exposed by the capping layer pattern. The method can also include forming linkers on the active layer exposed by the capping layer pattern, after the forming of the capping layer pattern, wherein the coupling of the capping layer pattern comprises coupling the capping layer pattern via the linkers. The capping layer pattern can be formed of a metallic film, a metallic nitride film, or a silicon nitride film. The bottom capping layer can be formed on the bottom surface of the substrate before the forming of the active layer.

In certain other embodiments, a method of fabricating a biochip can include forming a capping layer pattern partially covering a substrate on a top surface of the substrate, forming an active pattern on the top surface of the substrate exposed by the capping layer pattern, and coupling a plurality of probes to the active pattern. The method can also include forming linkers on the active pattern, after the forming of the active pattern, wherein the coupling of the plurality of probes comprises coupling the plurality of probes via the linkers. The capping layer pattern can be formed of a metallic film, a metallic nitride film, or a silicon nitride film. The bottom capping layer can be formed on the bottom surface of the substrate before the forming of the active pattern.

As described above, in biochips according to some embodiments of the disclosed technology and fabrication methods thereof, in non-probe cell regions between each of probe cell regions, unwanted coupling of linkers or probes to a bottom surface of a substrate can be prevented. In addition, in biochips according to other embodiments of the disclosed technology and fabrication methods thereof, an active layer or active patterns are selectively formed only on a top surface of the substrate without being formed on a bottom surface of the substrate. Accordingly, data noise can be suppressed, thereby improving the analysis reliability. Furthermore, in biochips according to other embodiments of the disclosed technology and fabrication methods thereof, use of a transparent substrate can increase analysis efficiency during fluorescence detection using a fluorescent material.

While the present invention has been particularly shown and described with reference to exemplary embodiments thereof, it will be understood by those of ordinary skill in the art that various changes in form and details may be made therein without departing from the spirit and scope of the present invention as defined by the following claims. It is therefore desired that the present embodiments be considered in all respects as illustrative and not restrictive, reference being made to the appended claims to indicate the scope of the invention.

What is claimed is:

1. A biochip comprising:
a substrate;
a capping layer pattern partially covering a top surface of the substrate; and
a plurality of probes coupled to the top surface of the substrate exposed by the capping layer pattern.

2. The biochip of claim 1, further comprising:
a plurality of probe cell regions to which the plurality of probes are coupled, respectively; and
a plurality of non-probe cell regions for isolating the respective probe cell regions from one another, to which the plurality of probes are not coupled, wherein the capping layer pattern is formed on the non-probe cell regions of the substrate, and the top surface of the substrate exposed by the capping layer pattern corresponds to a top surface of the plurality of probe cell regions.

3. The biochip of claim 1, further comprising linkers formed on the top surface of the substrate exposed by the capping layer pattern, and mediating coupling of the plurality of probes and the top surface of the substrate.

4. The biochip of claim 1, further comprising an active layer formed on substantially the entire top surface of the substrate, wherein the capping layer pattern is formed on the active layer and the plurality of probes are coupled to the active layer exposed by the capping layer pattern.

5. The biochip of claim 1, further comprising active patterns formed on the top surface of the substrate exposed by the capping layer pattern, wherein the plurality of probes are coupled to the active patterns.

6. The biochip of claim 1, wherein the capping layer pattern comprises a metallic film, a metallic nitride film, or a silicon nitride film.

7. The biochip of claim 1, further comprising a bottom capping layer formed on the bottom surface of the substrate.

8. The biochip of claim 7, wherein the bottom capping layer is formed of a metallic film, a metallic nitride film, or a silicon nitride film.

9. The biochip of claim 3, wherein the linkers are formed of a material containing functional groups.

10. The biochip of claim 9, wherein each of the functional groups comprises:
a first end coupled to the top surface of the substrate exposed by the capping layer pattern; and
a second end coupled to at least one of the plurality of probes.

11. A biochip comprising:
a substrate including a first region and a second region;
an active layer formed on the substrate; and
a probe cell isolating pattern formed on the active layer, wherein the probe cell isolating pattern is positioned on the second region.

12. The biochip of claim 11, wherein the probe cell isolating pattern is formed of a metallic film, a metallic nitride film, or a silicon nitride film.

13. The biochip of claim 11, wherein the active layer is formed on an entire top surface of the substrate.

14. The biochip of claim 11, further comprising a bottom capping layer formed on the bottom surface of the substrate.

15. The biochip of claim 14, wherein the bottom capping layer is formed of a metallic film, a metallic nitride film, or a silicon nitride film.

16. A biochip comprising:
a substrate including a first region and a second region;
a probe cell isolating pattern formed on the second region of the substrate; and
an active pattern formed on the first region of the substrate on the active layer, wherein a thickness of the active pattern is smaller than that of the probe cell isolating pattern.

17. The biochip of claim 16, wherein the probe cell isolating pattern is formed of a single film of a metallic film, a metallic nitride film, or a silicon nitride film.

18. The biochip of claim 16, wherein a top surface of the active pattern is lower than a top surface of the probe cell isolating pattern.

19. The biochip of claim 16, further comprising a bottom capping layer formed on the bottom surface of the substrate.

20. The biochip of claim 16, wherein the bottom capping layer is formed of a metallic film, a metallic nitride film, or a silicon nitride film.
